(19) United States
(12) Patent Application Publication
(10) Pub. No.: US 2006/0134026 A1
(43) Pub. Date: Jun. 22, 2006

(54) Sunscreens

(76) Inventors: George Barry Park, Oxford (GB); John Sebastian Knowland, Oxford (GB); Barry Richard Flutter, Oxford (GB)

Correspondence Address: WOLF GREENFIELD & SACKS, PC;FEDERAL RESERVE PLAZA, 600 ATLANTIC AVENUE, BOSTON, MA, 02210-2206, US

(21) Appl. No.: 10/540,649

(22) PCT Filed: Dec. 23, 2003
(86) PCT No.: PCT/GB03/05658

§ 371 Date: Jan. 3, 2006

(30) Foreign Application Priority Data
Dec. 24, 2002 (GB) 0230156.2
Apr. 1, 2003 (GB) 0307545.4
Apr. 15, 2003 (GB) 0308720.2
Jun. 27, 2003 (GB) 0315082.8

(51) Int. Cl. A61K 8/29; A61K 8/27

(52) U.S. Cl. 424/59; 977/926

(57) ABSTRACT

A UV sunscreening composition suitable for cosmetic or topical pharmaceutical use which comprises an amount of one or more organic components which are photosensitive and/or which are degraded and/or in which degradation is induced by another ingredient of the composition, and an amount of TiO₂ and/or ZnO which has been doped with another element and/or reduced zinc oxide, this composition having a rate of loss of UV absorption at least 5% less than that of a composition having the same formulation except that it does not contain the said TiO₂ and/or ZnO which has been doped with another element or the said reduced zinc oxide.

The present invention relates to UV screening compositions suitable for cosmetic and topical pharmaceutical use.

The effects associated with exposure to sunlight are well known. Thus exposure of the skin to UVA and UVB light may result in, for example, sunburn, premature ageing and skin cancer.

Commercial sunscreens generally contain components which are able to reflect and/or absorb UV light. These components include, for example, inorganic oxides such as zinc oxide and titanium dioxide as well as organic sunscreen agents.

The general public are generally more concerned by the obvious effects of sunlight, namely sunburn which causes reddening of the skin than they are with other effects of sunlight which are less self evident. As a consequence of this commercial sunscreen compositions are rated by a Sun Protection Factor (SPF). This is a measure of the time taken for skin to redden under a layer of the composition as compared with untreated skin. Thus an SPF of 20 indicates that skin will take 20 times longer to redden under a layer of the composition applied at 2 mg per cm² compared with untreated skin. This reddening effect is caused principally by UVB light. There is no recognised corresponding factor for the effects of UVA light even though the latter may be more damaging in the long term.

Most organic sunscreen agents absorb light over only a part of the UVA-UVB spectrum with the result that if one is to obtain a screening effect covering the whole UVA-UVB spectrum it is generally necessary to use a combination of different organic sunscreen agents. Some organic sunscreen agents and other components of sunscreen compositions are stable to UV light but others are photosensitive and/or may after being excited by UV light degrade, and/or induce degradation of, another ingredient of the composition.

Titanium dioxide and zinc oxide are generally formulated as “micronised” or “ultrafine” (20-50 nm) particles (so-called microreflectors) because particles whose size is less than 10% of the wavelength of the incident light scatter light according to Rayleigh's Law, whereby the intensity of scattered light is inversely proportional to the fourth power of the wavelength. Consequently, they scatter UVB light (with a wavelength of from 280 or 290 to 315/320 nm) and UVA light (with a wavelength of from 315/320 to 400 nm) more than the longer, visible wavelengths, preventing sunburn whilst remaining invisible on the skin.

However, titanium dioxide and zinc oxide also absorb UV light efficiently, leading via the initial formation of electron hole pairs to the formation of superoxide and hydroxyl radicals and which may in turn initiate damage to other components of the composition. The crystalline forms of TiO₂, anatase and rutile, are semiconductors with band gap energies of about 3.23 and 3.06 eV respectively, corresponding to light of about 385 nm and 400 nm (1 eV corresponds to 8066 cm⁻¹). Indeed there is evidence to suggest that TiO₂ can enhance the degradation of organic sunscreen agents, including UVA organic sunscreens, for example avobenzone. Attempts have been made to reduce the adverse effects of TiO₂ and ZnO by coating but coatings are not invariably effective.

The reason why most sunscreen agents do not have a substantially perpetual effect (i.e. an SPF factor which remains substantially constant) is principally because the organic sunscreen agents are degraded by light and/or are adversely affected by other components of the sunscreen composition once the latter are subjected to UV light.

It has now surprisingly been found, according to the present invention, that the degradation of organic sunscreen agents, and other components which are susceptible to degradation, can be retarded if the compositions also have present zinc oxide or titanium dioxide which has been doped with another element and/or reduced zinc oxide. In other words by using, in a cosmetic or topical pharmaceutical composition, these doped or reduced materials rather than ordinary titanium dioxide or zinc oxide it is, for example, possible either to provide a composition which gives better protection against UV light for the same quantity of organic sunscreen agent or a composition having the same screening effect against UV light but containing a smaller quantity of organic sunscreen agent. Indeed it is possible to provide all day protection sunscreens by incorporating the doped and/or reduced materials. Accordingly the present invention provides a cosmetic UV sunscreening composition suitable for cosmetic or topical pharmaceutical use which comprises an amount of one or more organic components which are photosensitive and/or which are degraded and/or in which degradation is induced by another ingredient of the composition and an amount of TiO₂ and/or ZnO which has been doped with one or more other elements, typically a second element, and/or reduced zinc oxide, this composition having a rate of loss of UV absorption at least 5% less than that of a composition having the same formulation except that it does not contain the said TiO₂ and/or ZnO which has been doped with another element or reduced zinc oxide. Thus if the rate of loss of UV absorption (during LTV exposure) over at least a proportion of the UVA and/or UVB spectrum is X then the amount of the organic component(s) which are photosensitive and/or which are degraded by another ingredient of the composition possesses a said rate of loss of Y where Y is greater than X by at least 5%, and the amount of doped TiO₂ and/or ZnO and/or reduced zinc oxide reduces the said rate of loss from Y to X.

The present invention also provides the use of a doped TiO₂/ZnO or reduced zinc oxide to reduce the concentration of one or more organic UV sunscreen or other photosensitive ingredient or ingredient which is degraded by another ingredient of the composition in a cosmetic UV screening composition as well as to reduce the rate of loss in UV absorption of a sunscreen composition containing one or more organic UV sunscreen agents. The present invention further provides a method of increasing the effectiveness (improve the stability) of an organic sunscreening composition which comprises one or more components which are photosensitive and/or which are degraded and/or in which degradation is induced by another ingredient of the composition which comprises incorporating into the composition a doped TiO₂/ZnO and/or reduced zinc oxide. Sometimes the degradation products (breakdown chemicals) are toxic. Accordingly, the present invention also provides a method of reducing the production of toxic compounds in a UV sunscreening composition which comprises incorporating therein a doped TiO₂/ZnO and/or reduced ZnO.

By “UV sunscreening composition suitable for cosmetic or topical pharmaceutical use” is meant any cosmetic or topical pharmaceutical composition having UV sunscreening activity i.e. it includes compositions whose principal function may not be sunscreening. The term “topical pharmaceutical” is used since, in some jurisdictions, the term “cosmetic” does not extend to compositions having a pharmaceutical effect. It will be appreciated that the doped TiO₂/ZnO or reduced ZnO may be the only ingredient of the composition having UV sunscreening activity i.e. the composition need riot necessarily contain an organic UV sunscreen agent. It is to be understood that the composition can also contain TiO₂ and/or ZnO which has not been doped or reduced.

The organic component which is photosensitive or may be degraded by another ingredient of the composition is generally a UV sunscreen agent. Although all organic sunscreen agents which suffer a loss in UV absorption can be used the present invention is particularly useful for agents which absorb in the UVA region as well as in the UVB region.

However, other organic components may also be susceptible to free radical attack with the degraded component potentially inducing degradation of the UV sunscreen agent.

As indicated above the UV absorption of an organic sunscreen agent generally decreases with time. In contrast the UV absorption of TiO₂ or ZnO does not decrease with time. Since TiO₂ and ZnO absorb in both the UVA and UVB region whereas an organic sunscreen agent is generally more wavelength specific it can be seen that the UVA/UVB absorption ratio may increase over time. When a doped TiO₂/ZnO is used rather than the same quantity of undoped TiO₂/ZnO the rate of change is reduced. This is because the doped material will enhance the performance of the organic sunscreen agent over time. Thus with a UVA sunscreen the loss of UVA absorption over time is reduced (i.e. the UVA response is more stable when the doped material is present) so that the ratio of change of the rates is reduced. Thus if the initial ratio of absorption is X/Y, it becomes

$$\frac{X - x}{Y} ,$$

where x is smaller when a doped material is used, with the result that the rate of change is less. With a UVB sunscreen, the rate of change is also reduced as a consequence of a more stable UVB response.

The rate of loss of absorption can be determined by illuminating a sample of the composition with and without the doped TiO₂ and/or ZnO of defined thickness with UV light of the appropriate wavelength and determining the absorption of UV by the composition over a given period, typically 60 minutes, obtaining a plot over that period for the wavelengths in question and determining the area under the curve from which the rate of loss can be calculated. Clearly the smaller the area under the curve the smaller the loss. For UVA absorption wavelengths from 320 to 400, especially from 340 to 390 nm, are considered.

While any reduction in the loss of UV absorption is an advantage, it is generally desirable that the presence of the doped oxide should reduce the rate of UV absorption by an amount of at least a 5%, preferably at least 10%, more preferably at least 15%, especially at least 20% and most preferably at least 40%.

A further feature of the present invention resides in the fact that the doped TiO₂/ZnO is generally coloured. As a result the use of such doped materials causes the composition to absorb more of the visible light which impinges upon it i.e. less visible light is transmitted and reaches the skin. Further in certain countries coloured formulations are advantageous. In skin lightening compositions in Japan, a pink colouration is useful in masking uneven colouration of the underlying skin. In Indonesia a yellow colouration can be seen as attractive. If, though, colour is to be minimised this can be achieved by coating the particles and/or by controlling the concentration of doping, both as discussed below.

The optimum amount of the other element in the host lattice may be determined by routine experimentation and in some embodiments is preferably low enough so that the particles are not coloured. Amounts as low as 0.1 mole % or less, for example 0.05 mole %, or as high as 1 mole % or above, for example 5 mole % or 10 mole %, can generally be used. Typical concentrations are from 0.5 to 2 mole % by weight.

The dopant for the oxide particles is preferably manganese, which is especially preferred, e.g. Mn²⁺ but especially Mn³⁺, vanadium, for example V^{3+ or V}⁵⁺, chromium and iron but other metals which can be used include nickel, copper, tin, aluminium, lead, silver, zirconium, zinc, cobalt, gallium, niobium, for example Nb⁵⁺, antimony, for example Sb³⁺, tantalum, for example Ta⁵⁺, strontium, calcium, magnesium, barium, molybdenum, for example Mo³⁺, Mo⁵⁺ or Mo⁶⁺ as well as silicon. Manganese is preferably present as Mn³⁺, cobalt as Co²⁺, tin as Sn⁴⁺ as well as Sn²⁺. These metals can be incorporated singly or in combination of 2 or 3 or more. Further details of these doped oxides can be found in WO99/60994 as well as WO01/40114.

These particles can be obtained by any one of the standard processes for preparing doped oxides and salts. Thus they can be obtained by a baking technique by combining particles of a host lattice (TiO₂/ZnO) with a second component in the form of a salt such as a chloride or an oxygen-containing anion such as a perchlorate or a nitrate, in solution or suspension, typically in solution in water, and then baking it, typically at a temperature of at least 300° C. Other routes which may be used to prepare the doped materials include a precipitation process of the type described in J. Mat. Sci. (1997) 36, 6001-6008 where solutions of the dopant salt and of an alkoxide of the host metal (Ti/Zn) are mixed, and the mixed solution is then heated to convert the alkoxide to the oxide. Heating is continued until a precipitate of the doped material is obtained. Further details of preparation can be found in the aforesaid patent specifications.

Doped TiO₂ or doped ZnO can also be obtained by flame pyrolysis or by plasma routes where mixed metal containing precursors at the appropriate level are exposed to a flame or plasma to obtain the desired product.

The rutile form of titania is known to be more photostable than the anatase form and is therefore preferred.

Reduced zinc oxide particles (i.e. particles which possess an excess of zinc ions relative to the oxygen ions) may be readily obtained by heating zinc oxide particles in a reducing atmosphere to obtain reduced zinc oxide particles which absorb UV light, especially UV light having a wavelength below 390 nm, and re-emit in the green, preferably at about 500 nm. It will be understood that the reduced zinc oxide particles will contain reduced zinc oxide consistent with minimising migration to the surface of the particles of electrons and/or positively charged holes such that when said particles are exposed to UV light in an aqueous environment the production of hydroxyl radicals is substantially reduced as discussed above.

The reducing atmosphere can be air with a reduced oxygen content or an increased hydrogen content but is preferably a mixture of hydrogen and an inert gas such as nitrogen or argon. Typically the concentration of hydrogen is from 1 to 20%, especially 5 to 15%, by volume, with the balance inert gas, especially nitrogen. A preferred reducing atmosphere is about 10% hydrogen and about 90% nitrogen by volume. The zinc oxide is heated in this atmosphere at, say, 5000 to 1000° C., generally 750 to 850° C., for example about 800° C., for 5 to 60 minutes, generally 10 to 30 minutes. Typically it is heated to about 800° C. for about 20 minutes.

It is believed that the reduced zinc oxide particles possess an excess of Zn²⁺ ions within the absorbing core. These are localised states and as such may exist within the band gap. A further discussion of this can be found in WO 99/60994.

The average primary particle size of the particles is generally from about 1 to 200 nm, for example about 1 to 150 nm, preferably from about 1 to 100 nm, more preferably from about 1 to 50 nm and most preferably from about 20 to 50 nm. The particle size is preferably chosen to avoid colouration of the final product. Thus nanoparticles are frequently used. However, in one embodiment slightly larger particles for example from 100 to 500 nm, typically 100 to 400 or 450 mm especially from 150 to 300 nm and particularly 200 to 250 nm, can be employed. These provide good coverage of, for example, skin imperfections without unacceptable skin whitening.

Where particles are substantially spherical then particle size will be taken to represent the diameter. However, the invention also encompasses particles which are non-spherical and in such cases the particle size refers to the largest dimension.

The oxide particles used in the present invention may have an inorganic or organic coating. For example, the particles may be coated with oxides of elements such as aluminium, zirconium or silicon, especially silica. The particles of metal oxide may also be coated with one or more organic materials such as polyols, amines, alkanolamines, polymeric organic silicon compounds, for example, RSi[{OSi(Me)₂}xOR¹]₃ where R is C₁-C₁₀ alkyl, R¹ is methyl or ethyl and x is an integer of from 4 to 12, hydrophilic polymers such as polyacrylamide, polyacrylic acid, carboxymethyl cellulose and xanthan gum or surfactants such as, for example, TOPO. Such coatings can have the effect of masking, at least to some extent, any colour which the doped particles may have.

The compositions of the present invention are generally for cosmetics use and may be, for example, lipsticks, skin anti-ageing compositions in the form of, for example, creams, including anti-wrinkle formulation exfoliating preparations including scrubs, creams and lotions, skin lightening compositions in the form of, for example, face powders and creams, preparations for the hands including creams and lotions, moisturising preparations, compositions for protecting the hair such as conditioners, shampoos and hair lacquers as well as hair masks and gels, skin cleansing compositions including wipes, lotions and gels, eye shadow and blushers, skin toners and serums as well as washing products such as shower gels, bath products including bubble baths, bath oils, but, preferably, sunscreens. In this connection we should point out that the expression “cosmetic UV sunscreening composition”, as used herein, includes any composition applied to the skin which may leave a residue on the skin such as some washing products. Compositions of the present invention may be employed as any conventional formulation providing protection from UV light. The compositions may also be pharmaceutical compositions suitable for topical application. Such compositions are useful, in particular, for patients suffering from disorders of the skin which are adversely affected by UV light such those giving rise to polymorphous light eruptions.

Organic sunscreen agents which can be used in the compositions of the present invention include any conventional sunscreen agent which gives protection against UV light while if there is no other photosensitive component the sunscreen agent is photosensitive and/or is degraded by another ingredient of the composition. Suitable sunscreen agents are listed in the IARC Handbook of Cancer Prevention, vol. 5, Sunscreens, published by the International Agency for Research on Cancer, Lyon, 2001 and include:
(a) Para-aminobenzoic acids (PABA), (UVB absorbers) esters and derivatives thereof, for example amyldimethyl-; ethyldihydroxypropyl-; ethylhexyl dimethyl-; ethyl-; glyceryl-; and 4-bis-(polyethoxy)-PABA.
(b) Cinnamates (UVB) especially esters including methyl cinnamate esters and methoxycinnamate esters such as octylmethoxy cinnamate, ethyl methoxycinnamate, especially 2-ethylhexyl para-methoxycinnamate, isoamyl p-methoxy cinnamate, or a mixture thereof with diisopropyl cinnamate, 2-ethoxyethyl-4-methoxycinnamate, DEA-methoxycinnamate (diethanolamine salt of para-methoxy hydroxycinnamate) or α,β-di-(para-methoxycinnamoyl)-α′-(2-ethylhexanoyl)-glycerin, as well as diisopropyl methylcinnamate;
(c) benzophenones (UVA) such as 2,4-dihydroxy-; 2-hydroxy-4-methoxy; 2,2′-dihydroxy-4,4′-dimethoxy-; 2,2′-dihydroxy-4-methoxy-;′ 2,2′,4,4′-tetrahydroxy-; and 2-hydroxy-4-methoxy-4′-methyl-benzophenones, benzenesulphoric acid and its sodium salt; sodium 2,2′-dihydroxy-4,4′-dimethoxy-5-sulphobenzophenone and oxybenzone;
(d) dibenzoylmethanes (UVA) such as butyl methoxydibenzoyl methane, especially 4-tert-butyl-4′methoxydibenzoylmethane;
(e) 2-phenylbenzimidazole-5 sulfonic acid UVB and phenyldibenzimidazole sulfonic acid and their salts;
(f) alkyl-β,β-diphenylacrytates (UVB) for example alkyl α-cyano-β,β-diphenylacrylates such as octocrylene;
(g) triazines (UVB) such as 2,4,6-trianilino-(p-carbo-2-ethyl-hexyl-1-oxy)-1,3,5 triazine as well as octyl triazone e.g. ethylhexyltriazone and diethylhexyl butamido triazone.
(h) camphor derivatives (generally UVB) such as 4-methylbenzylidene and 3-benzylidene-camphor and terephthalylidene dicamphor sulphonlic acid (UVA), benzylidene camphor sulphonic acid, camphor benzalkonium methosulphate and polyacrylamidomethyl benzylidene camphor;
(i) organic pigment sunscreening agents such as methylene bis-benzotriazole tetramethyl butylphenol;
(j) silicone based sunscreening agents such as dimethicodiethyl benzal malonate.
(k) salicylates (UVB) such as dipropylene glycol-; ethylene glycol-, ethylhexyl-, isopropylbenzyl-, methyl-, phenyl-, 3,3,5-trimethyl- and TEA-salicylate (compound of 2-hydroxybenzoic acid and 2,2′2″-nitrilotris (ethanol));
(l) anthranilates (UVA) such as menthyl anthranilate as well as bisymidazylate (UVA), dialkyl trioleate (UVB), 5-methyl-2-phenylbenzoxazole (UVB) and urocanic acid (UVB).

Some compounds are effective for both UVA and UVB. These include anisotriazine, methylene bisbenzotriazolyl tetramethylbutyl-phenol and drometrizole trisiloxane (Mexoryl XL).

The organic sunscreen agent(s) are typically present in the compositions at a concentration from 0.1 to 20%, preferably 1 to 10%, and especially 2 to 5%, by weight based on the weight of the composition.

In the compositions, which are generally aqueous, the metal oxides are preferably present at a concentration of about 0.5 to 20% by weight, preferably about 1 to 10% by weight and more preferably about 3 to 8% by weight, in particular about 4 to 7%, such as 4 to 6% for example about 5%, by weight.

The compositions may be in the form of, for example, lotions, typically with a viscosity of 4000 to 10,000 mPas, e.g. thickened lotions, gels, vesicular dispersions, creams, typically a fluid cream with a viscosity of 10,000 to 20,000 mPas or a cream of viscosity 20,000 to 100,000 mPas, milks, powders, solid sticks, and may be optionally packaged as aerosols and provided in the form of foams or sprays.

The compositions may contain any of the ingredients used in such formulations including fatty substances, organic solvents, silicones, thickeners, liquid and solid emollients, demulcents, other UVA, UVB or broad-band sunscreen agents, antifoaming agents, antioxidants such as butyl hydroxy toluene, buffers such as lactic acid with a base such as triethanolamine or sodium hydroxide, plant extracts such as Aloe vera, cornflower, witch hazel, elderflower and cucumber, activity enhancers, moisturizing agents, and humectants such as glycerol, sorbitol, 2-pyrrolidone-5-carboxylate, dibutylphthalate, gelatin and polyethylene glycol, perfumes, preservatives, such as para-hydroxy benzoate esters, surface-active agents, fillers and thickeners, sequesterants, anionic, cationic, nonionic or amphoteric polymers or mixtures thereof, propellants, alkalizing or acidifying agents, colorants and powders, including metal oxide pigments with a particle size of from 100 nm to 20000 nm such as iron oxides along with conventional (undoped) TiO₂ and ZnO.

Other ingredients of cosmetic compositions, for example some surface-active agents may have the effect of degrading certain sunscreen agents in the presence of UV light. Also TiO₂ and ZnO are known to degrade certain organic sunscreens such as avobenzone as well as antioxidants such as vitamins e.g. vitamins A, B, C and E and antiageing factors such as niacinamide, retenoids, coenzyme MEQ₁₀ etc. It will be appreciated that it is particularly useful to use the doped TiO₂ and/or ZnO and/or reduced ZnO with such sunscreens. This is because TiO₂ and ZnO do generally have a positive UV absorptive effect. Thus by using the doped TiO₂ and/or ZnO and/or reduced ZnO it may be possible to use less antioxidant or make the formulation longer lasting.

The organic solvents are typically from lower alcohols and polyols such as ethanol, isopropanol, propylene glycol, glycerin and sorbitol as well as methylene chloride, acetone, ethylene glycol monoethyl ether, diethylene glycol monobutyl ether, diethylene glycol mono-ethyl, ether, dimethyl sulphoxide, dimethyl formamide and tetrahydrofuran.

The fatty substances may consist of an oil or wax or mixture thereof, fatty acids, fatty acid esters, fatty alcohols, vaseline, paraffin, lanolin, hydrogenated lanolin or acetylated lanolin, beeswax, ozokerite wax and paraffin wax.

The oils are typically from animal, vegetable, mineral or synthetic oils and especially hydrogenated palm oil, hydrogenated castor oil, vaseline oil, paraffin oil, Purcellin oil, silicone oil such as polydimethyl siloxanes and isoparaffin.

The waxes are typically animal, fossil, vegetable, mineral or synthetic waxes. Such waxes include beeswax, Carnauba, Candelilla, sugar cane or Japan waxes, ozokerites, Montan wax, microcrystalline waxes, paraffins or silicone waxes and resins.

The fatty acid esters are, for example, isopropyl myristate, isopropyl adipate, isopropyl palmitate, octyl palmitate, C₁₂-C₁₅ fatty alcohol benzoates (“FINSOLV TN” from FINETEX), oxypropylenated myristic alcohol containing 3 moles of propylene oxide (“WITCONOL APM” from WITCO), capric and caprylic acid triglycerides (“MIGLYOL 812” from HULS).

The compositions may also contain thickeners such as cross-linked or non cross-linked acrylic acid polymers, and particularly polyacrylic acids which are cross-linked using a polyfunctional agent, such as the products sold under the name “CARBOPOL” by the company GOODRICH, cellulose, derivatives such as methylcellulose, hydroxymethylcellulose, hydroxypropyl methylcellulose, sodium salts of carboxymethyl cellulose, or mixtures of cetylstearyl alcohol and oxyethylenated cetylstearyl alcohol containing 33 moles of ethylene oxide.

Desirably, the weight ratio of water-dispersible titanium dioxide to oil-dispersible titanium dioxide is from 1:4 to 4:1, preferably from 1:2 to 2:1 and ideally about equal weight proportions.

Suitable emollients include stearyl alcohol, glyceryl monoricinoleate, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, eicosanyl alcohol behenyl alcohol, cetyl palmitate, silicone oils such as dimethylpolysiloxane, di-n-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rapeseed oil, safflower seed oil, evening primrose oil, soybean oil, sunflower seed oil, avocado oil, sesame seed oil, coconut oil, arachis oil, caster oil, acetylated lanolin alcohols, petroleum jelly, mineral oil, butyl myristate, isostearic acid, palmitic acid, isopropyl linoleate, lauryl lactate, myristyl lactate, decyl oleate, myristyl myristate.

Suitable propellants include propane, butane, isobutane, dimethyl ether, carbon dioxide, nitrous oxide.

Suitable powders include chalk, talc, fullers earth, kaolin, starch, gums, colloidal silica sodium polyacrylate, tetra alkyl and/or trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose, ethylene glycol monostearate.

When the compositions of the present invention are sunscreens they may be in the form of, for example, suspensions or dispersions in solvents or fatty substances or as emulsions such as creams or milks, in the form of ointments, gels, solid sticks or aerosol foams. The emulsions, which can be oil-in-water or water-in-oil emulsions, may further contain an emulsifier including anionic, nonionic, cationic or amphoteric surface-active agents; for a water-in-oil emulsion the HLB is typically from 1 to 6 while a larger value i.e >6 is desirable for an oil-in-water emulsion. Generally water amounts to up to 80%, typically 5 to 80%, by volume; Specific emulsifiers which can be used include sorbitan trioleate, sorbitan tristearate, glycerol monooleate, glycerol monostearate, glycerol monolaurate, sorbitan sesquioleate, sorbitan monooleate, sorbitan monostearate, polyoxyethylene (2) stearyl ether, polyoxyethylene sorbitol beeswax derivative, PEG 200 dilaurate, sorbitan monopalmitate, polyoxyethylen (3.5) nonyl phenol, PEG 200 monostearate, sorbitan monostearate, sorbitan monolaurate, PEG 400 dioleate, polyoxyethylene (5) monostearate, polyoxyethyene (4) sorbitan monostearate, polyoxyethylene (4) lauryl ether, polyoxyethylene (5) sorbitan monooleate, PEG 300 monooleate, polyoxyethylene (20) sorbitan tristearate, polyoxyethylene (20) sorbitan trioleate, polyoxyethylene (8) monostearate, PEG 400 monooleate, PEG 400 monostearate, polyoxyethylene (10) monooleate, polyoxyethylene (10) stearyl ether, polyoxyethylene (10) cetyl ether, polyoxyethylene (9.3) octyl phenol, polyoxyethylene (4) sorbitan monolaurate, PEG 600 monooleate, PEG 1000 dilaurate, polyoxyethylene sorbitol lanolin derivative, polyoxyethylene (12) lauryl ether, PEG 1500 dioleate, polyoxyethylene (14) laurate, polyoxyethylene (20) sorbitan monostearate, polyoxyethylene (20) sorbitan monooleate, polyoxyethylene (20) stearyl ether, polyoxyethylene (20) sorbitan monopalmitate, polyoxyethylene (20) cetyl ether, polyoxyethylene (25) oxypropylene monostearate, polyoxyethylene (20) sorbitol monolaurate, polyoxyethylene (23) lauryl ether, polyoxyethylene (50) monostearate, and PEG 4000 monostearate. Alternatively the emulsifier can be silicone surfactant, especially a dimethyl polysiloxane with polyoxyethylene and/or polyoxypropylene side chains, typically with a molecular weight of 10,000 to 50,000, especially cyclo-methicone and dimethicone copolyol. They may also be provided in the form of vesicular dispersions of ionic or nonionic amphiphilic lipids prepared according to known processes.

It can be advantageous to use both a water-dispersible and an oil-dispersible titanium dioxide or zinc oxide, at least one of which is doped or, in the case of zinc oxide, reduced. It has been found that when an emulsion is spread on the skin it has a tendency to break down into oily and non-oily areas. When the water evaporates the oil-dispersible particles will tend to be in the oily areas thus leaving areas unprotected. This can be avoided by having both hydrophilic and hydrophobic particles in the emulsion so that some are retained in hydrophilic areas and others in hydrophobic areas.

Water-dispersible particles can be uncoated or coated with a material to impart a hydrophilic surface property to the particles. Examples of such materials include aluminium oxide and aluminum silicate. Oil-dispersible particles which exhibit a hydrophobic surface property, are suitably coated with metal soaps such as aluminium stearate, aluminium laurate or zinc stearate, or with organosilicone compounds.

The following Examples further illustrate the present invention.

### EXAMPLE 1

The degradation of sunscreen formulations was assessed as follows:

Methods:

Preparation of Sample

Cut two strips of polythene 10 mm×25 mm and 12.5 microns thick.

Lay the polythene strips 20 mm apart on the centre of a quartz slide.

Pipette a drop of about 30 ml of sunscreen preparation onto the centre of the slide.

Carefully lay a second quartz slide on top of the sample and squeeze the slides together at the polythene strips thus providing a specimen 12.5 μm thick. Take care to avoid air bubbles.

Illumination

Use a Xenon lamp filtered with a Schott WG320 filter to carry out illuminations.

Take a base reading of light output using a spectroradiometer calibrated between 290 and 400 nm.

Measure the light intensity (290-400 nm) through a sample of water to use as a blank (Iq). The intensity over the range 290-400 nm is typical of that found in moderate latitudes in mid-summer.

Measure the light intensity (290-400 nm) through the sample (It) at time 0—as soon as it is put under the light—and then every 10 minutes for 1 hour.

At the end of the experiment take another base reading of light output to ensure that the light source has remained steady.

Calculations

Calculate the transmission (K) of the sunscreen film at each individual wavelength:
K=It/Iq
This can be used to plot wavelength vs transmission at each timepoint and shows the increase in transmission of an individual sunscreen during illumination.
The loss of light absorption (D) by the sunscreen at each individual wavelength is calculated as the proportion of the absorption of the sunscreen at T=0 still remaining at T=t:
D=K0/Kt
This can be used plot wavelength vs loss of light absorption. This plot allows comparisons to be made between different sunscreen preparations.
By measuring the area under this curve at each time point the rate of change of the total UVA absorption can also be calculated.
Formulations
Commercial sunscreens Factor 5 and Factor 10. These have the following ingredients.

The ingredients in italics are the active sunscreen agents.

These formulations were modified by the incorporation of doped and undoped TiO₂ and ZnO in various concentrations and compared with unmodified formulations.

Commercial Factor 5

Aqua

C12-15 alkyl benzoate

Glycerin

Butylene glycol dicaprylate/caprate

Ceteareth-20

Glyceryl stearate

Ehylhexyl triazone

Butyl methoxydibenzoylmethane

Disodium phenyl dibenzimidazole tetrasulfonate

PVP/hexadecane copolymer

Tocophenylacetate

Cetyl palmitate

Cetearyl alcohol

Ceteareth-12

Phenoxyethanol

Methylparaben

Ethylhexylglycerin

Trisodium EDTA

Sodium citrate

Citric acid

PEG-4 lauratePEG-4 dilaurate

PEG-4

Iodopropynyl butylcarbamate

Perfume

Commercial Factor 10

Aqua

Ethyl hexyl method cinnamate

Glycerin

Ceteareth-20

Butylene glycol dicaprylate/dicaprate

C12-15 alkyl benzoate

Glycerol stearate

Ethylhexyl triazone

Butyl metoxydibenzoylmethane

Phenoxyethanol

Cetyl palmitate

Cetearylatcohol

Ceteareth-12

PVP/hexadecane copolymer

Phenyl/benzimidazole sulphonate

Tocophenyl acetate

Methyl paraben

Ethythexylglycerin

Trisodium EDTA

PEG-4 laurate

PEG-4 dilaurate

PEG-4

Iodopropynyl butylcarbamate

BHT

Perfume

The results are shown in the attached Figures in which:

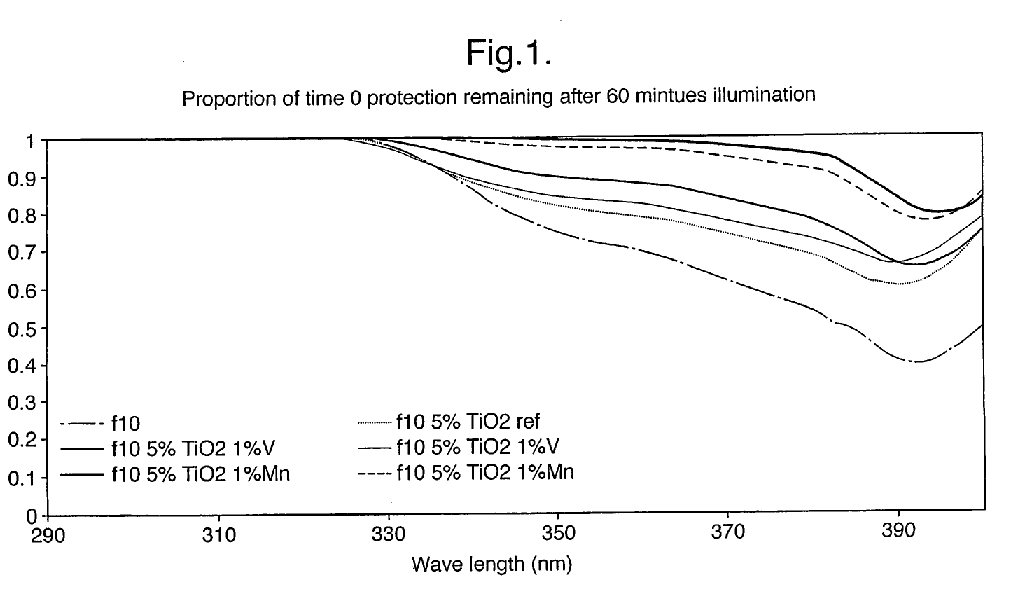

FIG. 1 shows the effect of time on absorption in UVA for a sunscreen formulation of factor 10 to which titanium dioxide, undoped or doped with 1% vanadium or manganese has been added.

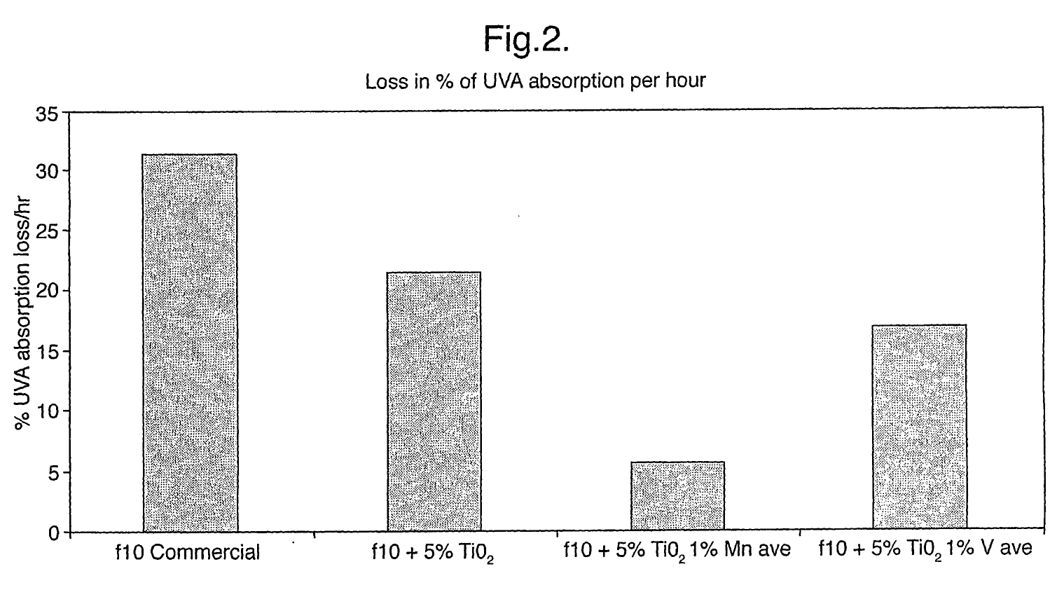

FIG. 2 gives the average percentage loss for several formulations.

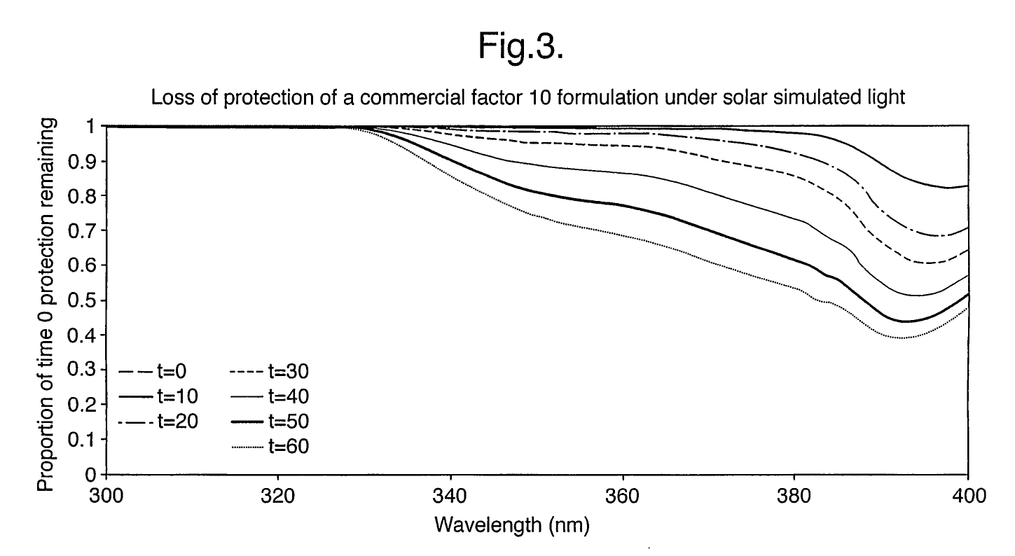

FIG. 3 shows the loss in absorption of a commercial factor 10 formulation in the UVA region at time=0 up to t=60.

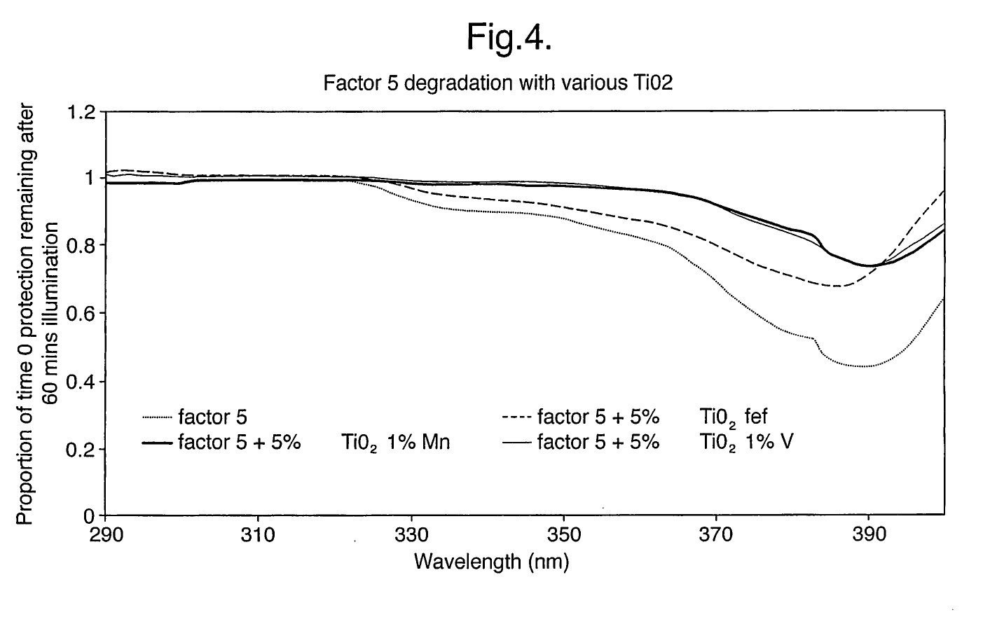

FIG. 4 shows the degradation of a factor 5 formulation to which has been added titanium dioxide doped or undoped.

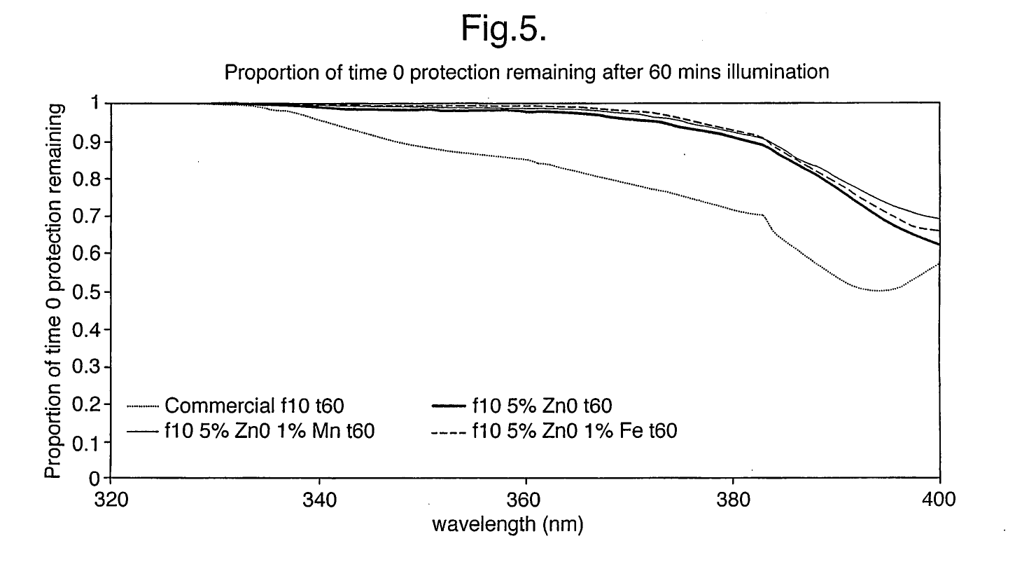

FIG. 5 shows the proportion of protection remaining for a commercial factor 10 formulation to which zinc oxide, undoped or doped with 1% manganese or iron has been added.

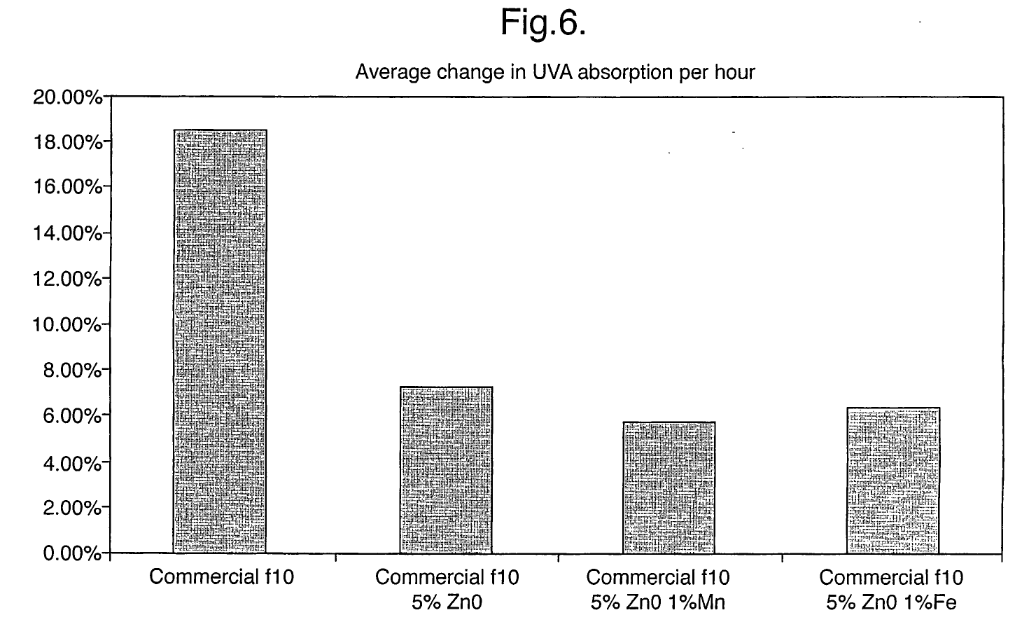

FIG. 6 shows the average change in UVA absorption of a commercial factor 10 formulation to which has been added zinc oxide undoped or doped with manganese or iron.

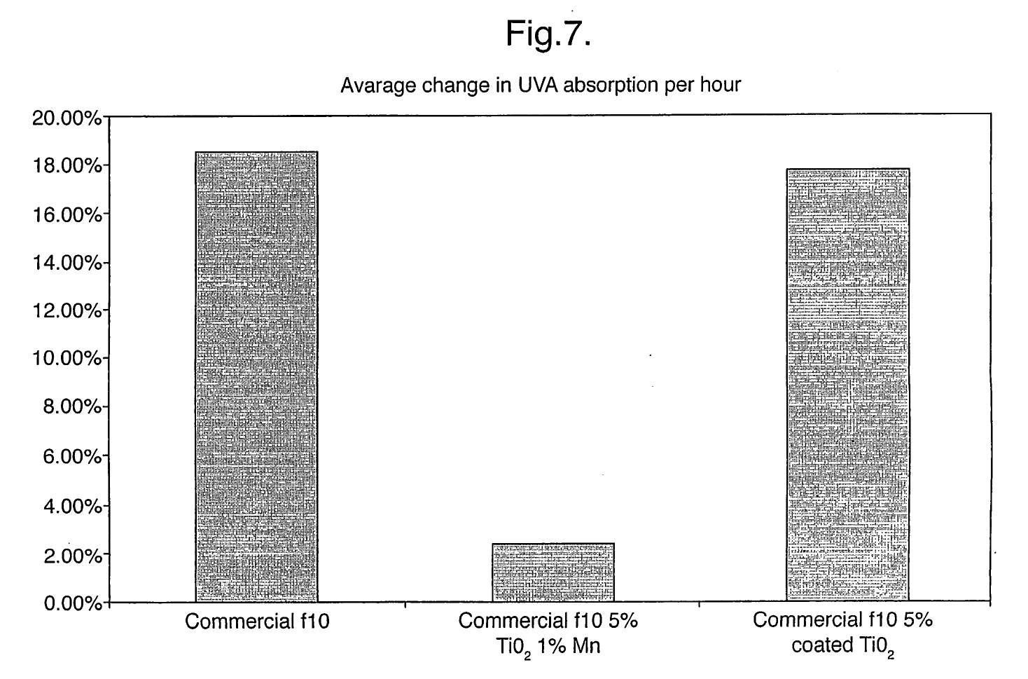

FIG. 7 shows the average change in UVA absorption for the same composition to which has been added TiO₂ which has been doped with manganese or coated; and

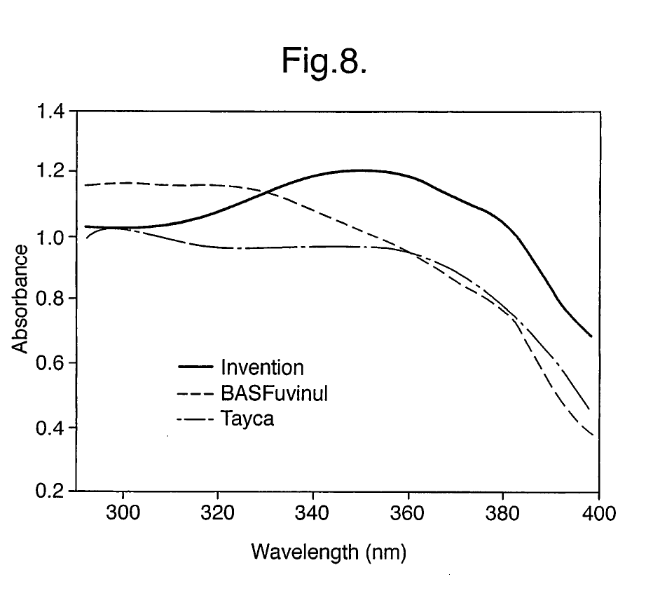

FIG. 8 compares absorption as a function of wavelength for the invention with that for two commercially-available compositions.

It can be seen that the addition of TiO₂ and ZnO reduces the rate of degradation, this being due partly to scattering and partly to additional absorption. TiO₂ or ZnO which is doped with manganese and vanadium, in particular, has a significantly greater effect in that the rate of loss of UV protection is reduced. Commercial Factor 10 was modified by the incorporation of ordinary or reduced ZnO of similar physical properties at 5% and compared. These materials were irradiated as described. The UVA absorption was recorded as a function of time to a total of 60 minutes. Each formulation containing reduced or ordinary zinc oxide showed about 2% transmission at time zero. The reduced zinc oxide however showed a reduced rate of loss of UVA absorption as a function of UV light exposure with a rate of loss of about 12% for ordinary zinc oxide and rate of loss of about 8% for reduced zinc oxide.

### EXAMPLE 2

A comparison was made between formulations differing solely in the nature of the TiO₂ incorporated; their absorbance was then measured.

The sunscreen formulations were based on a procedure by Stanley Black (www.sblack.com Formula Reference 1629).

| | % w/w |
|---|---|
| Phase A | |
| Water | 80.35 |
| Propylene Glycol | 2.00 |
| Methylparaben | 0.15 |
| Aloe Vera Gel ×1 | 0.10 |
| Phase B | |
| Lexemul 561 (Glyceryl Stearate, PEG-100 Stearate) | 5.00 |
| Lexemul GDL (Glyceryl Dilaurate) | 1.50 |
| Stearyl Alcohol NF | 0.30 |
| Lexol IPM (Isopropyl Myristate) | 1.00 |
| Lexol EHP (Octyl Palmitate) | 2.00 |
| Dow Corning 200 Fluid 200 cs (Dimethicone) | 0.50 |
| Propylparaben | 0.10 |
| Parsol 1789 (BMDM) | 2.00 |
| Titanium Dioxide | 5.00 |

They were prepared as follows:
Heat phase A to 75° C.
Heat phase A to 75° C.
Add phase A to phase B with vigorous stirring.
Cool to room temperature with stirring.
The TiO₂ was as follows:
A. TiO₂ doped with manganese to a level of approximately 1 mole %; primary particle size 20-30 nm; crystal form 99% rutile; no coating
B. Uvinul from BASF
Primary particle size—about 21 nm
Crystal form—75% Anatase/25% Rutile
Coating—Trimethylcaprylylsilane at 5%
C. MT100AQ from Tayca Corp
Primary particle size—15 nm
Crystal form—c.100% Rutile
Coating—Alumina/silica/alginic acid at up to 30%
The results obtained are shown in FIG. 8 which gives the degree of absorbance at different wavelengths for the 3 compositions. It is noticeable that the doped TiO₂ results in a significant increase in absorbance at around 360 nm, i.e. in the UVA region as compared with the two commercial undoped materials. In consequence by using such a doped TiO₂ it is possible to reduce the concentration of UVA sunscreen, organic or inorganic, in the formulation and/or to increase the effectiveness of the formulation against UVA radiation.

1. A UV sunscreening composition suitable for cosmetic or topical pharmaceutical use which comprises an amount of one or more organic components which are photosensitive and/or which are degraded and/or in which degradation is induced by another ingredient of the composition, and an amount of TiO₂ and/or ZnO which has been doped with one or more other elements and/or reduced zinc oxide, this composition having a rate of loss of UV absorption at least 5% less than that of a composition having the same formulation except that it does not contain the said TiO₂ and/or ZnO which has been doped with another element or the said reduced zinc oxide.

2. A composition according to claim 1 which is suitable for cosmetic use.

3. A composition according to claim 1 which contains TiO₂ and/or ZnO which has not been doped or reduced.

4. A composition according to claim 1 wherein the dopant is manganese, vanadium, chromium or iron.

5. A composition according to claim 4 wherein the dopant is Mn³⁺.

6. A composition according to claim 1 wherein the dopant is present in an amount from 0.05% to 10 mole %.

7. A composition according to claim 6 wherein the dopant is present in an amount from 0.5 to 2 mole % by weight.

8. A composition according to claim 1 which comprises doped titanium dioxide.

9. A composition according to claim 8 wherein the titanium dioxide is in rutile form.

10. A composition according to claim 1 which comprises reduced zinc oxide.

11. A composition according to claim 1 which comprises 0.5 to 20 mole % by weight of the doped TiO₂ or ZnO or reduced ZnO.

12. A composition according to claim 1 wherein the doped or reduced oxide has a particle size from 1 to 200 nm.

13. A composition according to claim 1 wherein the doped or reduced oxide has a particle size from 100 to 500 nm.

14. A composition according to claim 1 wherein one or more of the said organic components is a UV sunscreen agent.

15. A composition according to claim 14 wherein the organic sunscreen agent absorbs UV light in the UVA region.

16. A composition according to claim 14 wherein the organic sunscreen agent is a paraminobenzoic acid, ester or derivative thereof, a methoxy cinnamate ester, a benzophenone, a dibenzylomethane, an alkyl-β,β-phenyl acrylate, a triazine, a camphor derivative, an organic pigment, a silicone based sunscreen agent or 2-phenylbenzimdazoyl-5 sulphonic acid or phenyldibenzimidazoyl sulphonic acid.

17. A composition according to claim 1 wherein the said rate of loss of UV absorption is a rate of loss of UVA absorption.

18. A composition according to claim 1 wherein the rate of change of the ratio of the loss of UVA absorption to the loss of UVB absorption is less than that of a composition of the same formulation except that the TiO₂ and/or ZnO present is not doped.

19. A composition according to claim 18 wherein the rate of change of the ratio is greater because the rate of loss of UVA absorption is reduced.

20. A composition according to claim 1 which comprises 0.1% to 20% by weight of organic sunscreen agent(s).

21. A composition according to claim 1 which contains one or more of a fatty substance, organic solvent, silicone, thickener, demulcents, UVB sunscreen agent, antifoaming agent, moisturising agent, perfume preservative, surface activation filler, sequestrant, anionic, cationic, nonionic or amphoteric polymer, propellant, alkalising or acidifying agent, colorant or metal oxide pigment.

22. A composition according to claim 1 which is a sunscreen.

23. A composition according to claim 1 which is in the form of a lotion, gel, dispersion, cream, milk, powder or solid stick.

24. A composition according to claim 23 which comprises a water-dispersible and an oil-dispersible TiO₂ and/or ZnO.

25. A composition according to claim 1 wherein the TiO₂ and/or ZnO is coated with an inorganic or organic solvent.

26. (canceled)

27. A method for reducing the concentration of one or more organic UV sunscreen agents or other ingredients which is photosensitive and/or is degraded and/or in which degradation is induced by another ingredient in a cosmetic UV screening composition, comprising incorporating into the composition a doped or reduced TiO₂/ZnO as defined in claim 1.

28. A method for reducing the rate of loss in UV absorption of a sunscreen composition, comprising incorporating into the composition a doped or reduced TiO₂/ZnO as defined in claim 1.

29. A method for reducing the rate of change of the ratio of the loss of UVA absorption to the loss of UVB absorption in a cosmetic UV screening composition, comprising incorporating into the composition a doped or reduced TiO₂/ZnO as defined in claim 1, wherein the composition comprises one or more organic components which are photosensitive and/or which are degraded by another ingredient of the composition in a relation a composition of the same formulation except that the TiO₂ and/or ZnO present is not doped or reduced.

30. A method of increasing the effectiveness of an organic UV sunscreening composition, which comprises one or more components which are photosensitive and/or are degraded and/or in which degradation is induced by another ingredient of the composition which comprises incorporating into the composition a doped or reduced TiO₂/ZnO as defined in claim 1.

31. A method of reducing the production of a toxic compound in a UV sunscreening composition which comprises incorporating therein doped TiO₂ and/or doped or reduced ZnO as defined in claim 1.
